(19) United States
(12) Patent Application Publication
(10) Pub. No.: US 2004/0166095 A1
(43) Pub. Date: Aug. 26, 2004

(54) Methods for preventing GVHD

(76) Inventors: Bruce R. Blazar, Golden Valley, MN (US); Matthew J. O'Shaughnessy, Falcon Heights, MN (US); Christine Vogtenhuber, Minneapolis, MN (US); Jonathan S. Serody, Chapel Hill, NC (US); Albert S. Baldwin, Chapel Hill, NC (US); Brian Poligone, Palo Alto, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/374,222

(22) Filed: Feb. 25, 2003

(51) Int. Cl. A61K 45/00; C12N 5/08

(52) U.S. Cl. 424/93.7; 435/372

(57) ABSTRACT

Methods are provided for administering alloreactive T cells to transplant recipients such that the risk of graft-versus-host disease is reduced.

### TECHNICAL FIELD

This invention relates to methods for treating transplanted allogeneic or xenogeneic tissue or organs ex vivo in order to tolerize T cells contained therein to recipient antigens. The invention also relates to methods for treating a transplant recipient so that allogeneic or xenogeneic donor tissue or organs are tolerized in vivo to recipient antigens. The methods provided herein can be used to reduce the risk of graft-versus-host disease.

### BACKGROUND

Graft-versus-host disease (GVHD) is a possible complication of any transplant that uses stem cells from either a related or an unrelated donor. Such transplants typically are used in the treatment of disorders such as leukemia, bone marrow failure syndromes, and inherited disorders (e.g., sickle cell anemia, thalassemia, immunodeficiency disorders, and metabolic storage diseases such as mucopolysaccharidosis), as well as low-grade lymphoma. GVHD arises from a reaction of donor T lymphocytes against major histocompatibility complex (MHC) or minor histocompatibility antigen disparities present on antigen-presenting cells (APC) and various tissues of the individual receiving the donor cells. GVHD can be exacerbated by tissue injury induced by pre-bone marrow transplant conditioning that includes destruction of the recipient's bone marrow.

Acute GVHD usually occurs within the first three months following a transplant, and can affect the skin, liver, stomach, and/or intestines. Chronic GVHD is the late form of the disease, and usually develops three months or more after a transplant. The symptoms of chronic GVHD resemble spontaneously occurring autoimmune disorders such as lupus or scleroderma.

### SUMMARY

The invention is based on the discovery that inhibition of NF-κB activation in alloreactive T cells can result in hyporesponsiveness and a reduced risk of GVHD. The present invention thus provides an effective means of preventing or inhibiting GVHD responses that could otherwise occur upon transplantation of donor T cells or tissues or organs containing T cells (e.g., bone marrow or peripheral blood cells) into a recipient. Donor T cells can be incubated ex vivo with cells from the transplant recipient and a sufficient amount of an inhibitor of NF-κB activation, for a sufficient time to render the donor T cells substantially non-responsive to recipient cells upon transplantation. Alternatively, a transplant recipient can be treated with an inhibitor of NF-κB inhibition in vivo, either prior to, concurrent with, or after transplantation.

The methods provided herein have significant potential in the area of bone marrow or peripheral blood cell transplantation therapies. Bone marrow and stem cell transplantation is conventionally utilized for treatment of diseases such as leukemia and other conditions involving immune cell deficiencies. Bone marrow transplantation also may afford benefits in the treatment of other diseases (e.g., autoimmune diseases). However, a prevalent risk associated with conventional allogeneic bone marrow transplantation therapy is the risk of eliciting a GVHD response. The methods described herein provide tremendous potential in the treatment of transplant recipients since they afford a highly efficient, non-invasive means of rendering transplanted T cells tolerized or non-responsive to recipient alloantigens or xenoantigens. Consequently, transplanted tissues or organs (e.g., allogeneic or xenogeneic bone marrow) should not elicit an adverse graft-versus-host response upon transplantation. Methods of the invention thus are useful to reduce or even eliminate the risk of GVHD and thereby extend the clinical indications for bone marrow transplantation therapies.

In one aspect, the invention features a method for administering hematopoietic stem cells to a transplant recipient. The method can include: (a) providing a donor hematopoietic stem cell composition containing non-autologous T cells; (b) contacting the composition with an inhibitor of NF-κB activation to induce hyporesponsiveness of the T cells; and (c) administering the contacted composition to the recipient. The contacted composition can have a lower GVHD potential than a corresponding non-contacted composition. The non-autologous T cells can be alloreactive T cells. The inhibitor of NF-κB activation can be PS1145.

In another aspect, the invention features a method for administering hematopoietic stem cells to a transplant recipient. The method can include administering to the recipient (a) an inhibitor of NF-κB activation, and (b) a donor hematopoietic cell composition containing alloreactive T cells. The inhibitor of NF-κB activation can be PS1145. The inhibitor can be administered prior to the transplant, concurrent with the transplant, or after the transplant. The inhibitor can be administered orally or parenterally. The recipient can have cancer (e.g., a cancer that results in a solid tumor or a hematopoictic cancer). The recipient can have a bone marrow failure syndrome or an inherited disorder.

In another aspect, the invention features a method of making a T cell tolerized to an alloantigen. The method can include providing an isolated T cell, and contacting the isolated T cell with the alloantigen and an inhibitor of NF-κB activation.

In still another aspect, the invention features a method for treating an autoimmune disorder in a subject. The method can involve identifying a subject having an autoimmune disorder, and administering to the subject a composition containing a direct inhibitor of IKK. The inhibitor can be PS1145.

The invention also features a method for reducing transplant rejection. The method can include administering to a transplant recipient a composition containing a direct inhibitor of IKK, wherein the transplant is an organ transplant. The inhibitor can be PS1145.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph plotting allogen-induced proliferation of T cells in primary mixed lymphocyte reaction (MLR) cultures that were treated with anti-CD40L monoclonal antibody or left untreated.

FIG. 2 is a graph plotting survival of mice injected with cells from primary MLR cultures that were treated with anti-CD40L monoclonal antibody or left untreated.

FIG. 3 is a graph plotting survival of mice injected with cells from secondary MLR cultures that were treated with anti-CD40L monoclonal antibody or left untreated.

FIG. 4 is a graph plotting IL-2 and IFN-γ levels in MLR cultures treated with anti-CD40L monoclonal antibody or left untreated.

FIG. 5 is a graph plotting allogen-induced proliferation of T cells in MLR cultures treated with anti-CD40L monoclonal antibody and exposed to exogenous IL-2 or vs. proliferation in untreated MLR cultures.

FIG. 6 is a graph plotting allogen-induced proliferation of T cells in MLR cultures treated with the indicated amounts of PS1145, DMSO, or left untreated.

FIG. 7 is a graph plotting allogen-induced proliferation of T cells in MLR cultures treated with vehicle, treated with PS1145, or treated with PS1145 and exposed to exogenous IL-2.

FIG. 8 is a graph plotting proliferation of T cells in MLR cultures treated with vehicle or PS1145 and exposed to allogeneic APC, CD3+ and CD28+cells, or exogenous IL-2.

FIG. 9 is a graph plotting allogen-induced proliferation of T cells in various MLR cultures treated with PS1145 or left untreated.

FIG. 10 is a graph plotting LPS-induced expression of TNF-α and MIP-1α in mice treated with the indicated amounts of PS1145.

FIG. 11 is a graph plotting survival of mice treated in vivo with PS 145 beginning on the indicated days relative to bone marrow transplantation.

FIG. 12 is a graph plotting survival of mice orally treated with PS1145 or methylcellulose prior to bone marrow transplantation.

FIG. 13 is a graph plotting levels of TNF-α in mice treated with PS1145 or left untreated.

FIG. 14 is a graph plotting levels of MIP-1α in mice treated with PS 145 or left untreated.

### DETAILED DESCRIPTION

The present invention provides methods for preventing conditions that can arise from the introduction of foreign hematopoeitic stem cells into a subject (e.g., an immunocompromised subject receiving a bone marrow transplant). Such conditions include GVHD, in which donor T cells attack cells within a transplant recipient. The methods provided herein are useful to lower “GVHD potential,” which refers to the propensity for T cells to cause a graft-versus-host response upon transplantation into a recipient. Thus, the methods described herein can result in prevention or inhibition of GVHD responses that might otherwise occur upon transplantation of tissues or organs containing T cells (e.g., bone marrow or peripheral blood cells) into a recipient.

As described below, donor T cells can be incubated ex vivo with cells from the transplant recipient and a sufficient amount of an inhibitor of NF-κB activation, for a sufficient time to render the donor T cells substantially non-responsive to recipient cells upon transplantation. Alternatively, a transplant recipient can be treated with an inhibitor of NF-κB activation in vivo, either prior to, concurrent with, or after transplantation. Methods of the invention can be useful for treating individuals having, for example, leukemia or another hematopoeitic cancer, a bone marrow failure syndrome, an autoimmune disorder, or a cancer that results in a solid tumor (e.g., renal cell carcinoma, soft tissue sarcoma, melanoma, and breast cancer).

Methods of Preventing GVHD

According to the methods provided herein, donor hematopoeitic stem cells are contacted with a composition containing an inhibitor of NF-κB activation. NF-κB is a transcription factor that plays roles in such diverse processes as immunity, inflammation, infection, apoptosis, cell growth, and differentiation. The active form of NF-κB is a heterodimer of 50 kDa and 65 kDa subunits. This heterodimer is sequestered in the cytoplasm by one of several inhibitory factors known as IκB. Phosphorylation of IκB by a specific kinase (IKK) targets the inhibitor for ubiquitination and degradation, releasing active NF-κB for entry into the nucleus where it can activate the expression of genes encoding adhesion molecules, cytokines, acute phase proteins, anti-apoptosis proteins, and chemokines, for example. Compositions used in the methods provided herein thus result in reduced expression of genes normally activated by NF-κB (e.g., genes encoding cytokines such as IL-2, MIP-1α, and TNF-α).

Compounds that are useful in the methods provided herein can inhibit activation of NF-κB either directly or indirectly. For example, a compound can act directly on NF-κB to prevent the interaction between the 50 kDa and 65 kDa subunits or to prevent dissociation of IκB from the NF-κB complex. Alternatively, a compound can indirectly prevent activation of NF-κB by, for example, inhibiting phosphorylation of IκB by IKK. The compound PS1145 (Millennium Pharmaceuticals, Cambridge, Mass.), which inhibits IKK, is an example of an indirect inhibitor of NF-κB activation. Thus, compounds that are useful in the methods provided herein include direct inhibitors of NF-κB activation and direct inhibitors of IKK activity, for example. It is to be understood, however, that a direct inhibitor of IKK such as PS1145 also is an indirect inhibitor of NF-κB activation.

Methods of the invention can include setting up a mixed lymphocyte reaction (MLR), in which a preparation of donor hematopoeitic stem cells that includes alloreactive T cells is mixed with cells from a recipient that is allogeneic or xenogeneic to the donor. As used herein, a “hematopoeitic stem cell” is a cell capable of differentiating into multiple types or even all types of blood cells. Such stem cells typically are found in bone marrow together with other cell types such as T cells, although isolation from the circulation is possible using appropriate separation techniques. An “allogeneic cell” refers to a cell obtained from a different individual of the same species as the recipient, and “alloantigen” refers to an antigen expressed by a cell obtained from a different individual of the same species as the recipient. Thus, an “alloreactive T cell” is a T cell that exhibits an immune response to an alloantigen. As used herein, “xenogeneic cell” refers to a cell obtained from a different species relative to another species, and “xenoantigen” refers to an antigen expressed by a cell obtained from a different species relative to another species. For example, baboon T cells would comprise xenogeneic cells if transplanted into a human recipient. A “non-autologous cell” is a cell obtained from either a different individual of the same species as the recipient or from a different species. Thus, non-autologous cells include both allogeneic and xenogeneic cells.

As used herein, an “isolated cell” is a cell that has been removed from the organism in which it would normally be found. For example, a red blood cell in a blood sample obtained from a particular individual is considered an isolated red blood cell. Similarly, a T cell in a bone marrow sample obtained from an individual is an isolated T cell. Thus, an isolated cell can be contained within a population of different cell types. In other embodiments, an isolated cell can be contained within a population of other cells of the same type, or can be separated from all other cells altogether. A cell line propagated in culture also is considered to contain “isolated cells.”

A very small proportion of donor T cells possess the capability to recognize host alloantigen. Methods of the invention can be used to eliminate this response and thus render such cells non-responsive or hyporesponsive (i.e., tolerized) to alloantigen or xenoantigen by functionally altering the population of T cells with alloantigen or xenoantigen reactive capabilities. As used herein, “T cell non-responsiveness” or “hyporesponsiveness” refers to the reduced immune response (graft-versus-host response) elicited by donor T cells against cells bearing alloantigen or xenoantigen upon transplantation of the donor T cells into a recipient. Such non-responsiveness or hyporesponsiveness typically occurs after donor T cells have been contacted ex vivo or in vivo with alloantigen- or xenoantigen-bearing cells and an inhibitor of NF-κB activation.

The fact that tolerance can be induced ex vivo is advantageous as this treatment can be utilized in conjunction with other anti-rejection strategies such as those employing cyclosporine or other immunosuppressants. These other anti-rejection reagents can be administered prior to, concurrent with, or subsequent to transplantation of alloreactive donor T cells contacted ex vivo with an inhibitor of NF-κB activation. Alternatively, the subject method may eliminate the need for other immunosuppressant anti-rejection drugs, which can have adverse side effects (e.g., increased risk of infection or cancer).

In one embodiment, T cells from a donor (e.g., an allogeneic or xenogeneic donor) can be cultured ex vivo with recipient allogeneic or xenogeneic tissue that has been treated with, for example, irradiation. To this MLR culture, an effective amount of an inhibitor of NF-κB activation can be added. Suitable inhibitors of NF-κB activation include the compounds PS1145 and PS-341 (Millennium Pharmaceuticals), as well as thalidomide and bortezomib, for example. The MLR culture can be maintained for a time sufficient to induce T cell hyporesponsiveness. Typically, this time will range from about 1 or 2 days to about 30 days, (e.g., 3-15 days, 4-12 days, or 5-7 days). After culturing, the donor T cells can be tested to determine whether they elicit an anti-host alloantigen or xenoantigen response. Also, it can be determined whether such cells remain viable and otherwise elicit normal T cell activity after treatment. As shown in the Examples below, donor T cells treated in this way exhibited markedly blunted anti-host xenoantigen or alloantigen responses, and maintained viability. Also, upon restimulation, these donor T cells maintained their anti-host alloantigen hyporesponsiveness. It was further observed that in primary MLR, the production of T-helper type 1 (Th1) cytokines was markedly reduced. Similarly, Th1 cytokine production was markedly reduced in secondary restimulation cultures.

Moreover, it was found that administration to mice of either untreated alloreactive donor T cells or alloreactive donor T cells treated ex vivo with PS1145 resulted in markedly different GVHD potentials. Specifically, recipients of donor T cells treated ex vivo with PS1145 had a 78% long-term survival rate as compared to 0% of recipients of untreated donor cells (see Example 2). Thus, donor T cells can be effectively tolerized ex vivo in a MLR. This provides an important new approach for invoking donor T cell tolerization to host cells and xenoantigens.

In addition to methods for treating bone marrow or peripheral blood cells ex vivo as described above, the invention provides methods for treating a transplant recipient in vivo with an inhibitor of NF-κB activation. The inhibitor can be administered to a recipient prior to, concurrent with, or subsequent to introduction of transplanted cells (e.g., bone marrow or peripheral blood cells). Administration of an inhibitor prior to transplantation is particularly useful. Typically, an inhibitor is administered in an amount that is effective to inhibit the activation of NF-κB in donor cells or tissues contacted by the inhibitor. As active NF-κB stimulates expression of a wide variety of genes encoding, for example, cytokines such as IL-2, MIP-1α, IL-1α, IL-1β, IL-6, IL-12, and TNF-α, the effectiveness of an inhibitor can be monitored by measuring the expression of one or more of these genes. The ability of a compound to inhibit expression and/or production of a cytokine such as IL-2 can be assessed by measuring levels of IL-2 mRNA or protein in a transplant recipient before and after treatment, for example. It is noted that such methods also can be used to monitor the effectiveness of an inhibitor on donor cells treated ex vivo with an inhibitor of NF-κB activation. Methods for measuring mRNA and protein levels in cells, tissues, and biological samples are well known in the art. If the subject is a research animal, for example, IL-2 levels in a particular tissue (e.g., liver) can be assessed by in situ hybridization or immunostaining following euthanasia. Indirect methods can be used to evaluate the effectiveness of an inhibitor in live subjects. For example, reduced expression of a cytokine such as IL-2 can be inferred from a lack of inflammation after transplant. As with the ex vivo methods, in vivo methods of inducing donor T cell hyporesponsiveness can be combined with other anti-rejection treatments such as, for example, in vivo infusion of immunosuppression agents such as methatrycide, cyclosporine A, or steroids.

Ideally, the methods provided herein will provide for immune reconstitution in a recipient of the treated donor T cells without eliciting any graft-versus-host response. In some instances, however, this therapy may need to be repeated if the transplanted tissue does not “take” in the transplant recipient. Alternatively, repeat treatment may be necessary if the lymphoid system of the recipient becomes impaired again as a result of disease or treatment or the disease (e.g., subsequent radiation treatment). In such cases, suitable donor T cells can again be contacted ex vivo with T cell depleted alloantigen- or xenoantigen-bearing recipient cells and an inhibitor of NF-κB activation to induce T cell hyporesponsiveness, and then administered to the transplant recipient. Alternatively, the recipient can be treated in vivo with an inhibitor of NF-κB activation and infused with donor T cells.

Compositions for Inducing Alloreactive T Cell Hyporesponsiveness

An inhibitor of NF-κB activation can be used for the preparation of a medicament or pharmaceutical composition for use in any of the methods of treatment provided herein. Thus, an inhibitor of NF-κB activation can be combined with an appropriate diluent or pharmaceutically acceptable carrier for administration to a donor T cell preparation (e.g., a MLR) or to a transplant recipient. Such pharmaceutical compositions can be administered in amounts and for periods of time that will vary depending upon the nature of the type of transplant and the subject's overall condition. Methods for formulating and subsequently administering pharmaceutical compositions are well known to those skilled in the art. Dosing generally is dependent on the severity and responsiveness of the disease state to be treated, with the course of treatment lasting from several days to several months, or until a cure is effected or a diminution of the disease state is achieved. Persons of ordinary skill in the art can routinely determine optimum dosages, dosing methodologies, and repetition rates. Optimum dosages can vary depending on the relative potency of individual inhibitor compounds, and generally can be estimated based on EC₅₀ found to be effective in in vitro or ex vivo cell models and in vivo animal models. Typically, dosage is from 0.01 μg to 100 g per kg of body weight. A composition can be given once or more daily, weekly, monthly, or even less often.

Useful pharmaceutical compositions and formulations can include inhibitors of NF-κB activation (e.g., PS1145). An inhibitor such as PS1145 can be admixed, encapsulated, conjugated or otherwise associated with other molecules, molecular structures, or mixtures of molecules such as, for example, liposomes, receptor targeted molecules, or oral, rectal, topical or other formulations, for assisting in uptake, distribution and/or absorption.

A “pharmaceutically acceptable carrier” is a pharmaceutically acceptable solvent, suspending agent, or any other pharmacologically inert vehicle for delivering one or more compounds (e.g., PS1145) to a donor T cell ex vivo or to a T cell transplant recipient. Pharmaceutically acceptable carriers can be liquid or solid, and can be selected with the planned manner of administration in mind so as to provide for the desired bulk, consistency, and other pertinent transport and chemical properties, when combined with one or more compounds and any other components of a given pharmaceutical composition. Typical pharmaceutically acceptable carriers include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

Pharmaceutical compositions can be administered by a number of methods depending upon whether local or systemic treatment is desired. Systemic treatment typically is desired for prevention of GVHD in a transplant recipient. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, a composition can be administered by injection or infusion into the cerebrospinal fluid, preferably with one or more agents capable of promoting penetration of the inhibitor across the blood-brain barrier.

Formulations for topical administration of inhibitors of NF-κB activation include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions often are biphasic systems comprising of two immiscible liquid phases intimately mixed and dispersed with each other; in general, emulsions are either of the water-in-oil (w/o) or oil-in-water (o/w) variety. Emulsion formulations are particularly useful for oral delivery of therapeutics due to their ease of formulation and efficacy of solubilization, absorption, and bioavailability.

Compositions useful for inhibiting NF-κB activation can further encompass any pharmaceutically acceptable salts, esters, or salts of such esters, or any other compound which, upon administration to an animal including a human, is capable of providing (directly or indirectly) the biologically active metabolite or residue thereof. Accordingly, for example, it is possible to use pharmaceutically acceptable salts of inhibitors such as PS1145, prodrugs and pharmaceutically acceptable salts of prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the inhibitors of NF-κB activation useful in methods of the invention (i.e., salts that retain the desired biological activity of the parent inhibitor compound without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed from elemental anions (e.g., chlorine, bromine, and iodine).

Certain embodiments of the invention provide pharmaceutical compositions containing (a) one or more inhibitors of NF-κB activation, and (b) one or more other agents that function by a different mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions of the invention. Other agents (e.g., chemotherapeutic agents) also can be included within the compositions. Such combined compounds can be used together or sequentially.

Compositions useful in the methods of the present invention additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the inhibitors within the compositions provided herein.

Pharmaceutical formulations can be presented conveniently in unit dosage form, and can be prepared according to conventional techniques well known in the pharmaceutical industry. Such techniques include the step of bringing into association the active ingredients (e.g., one or more inhibitors of NF-κB activation) with the desired pharmaceutical carrier(s) or excipient(s). Typically, the formulations can be prepared by uniformly bringing the active ingredients into intimate association with liquid carriers or finely divided solid carriers or both, and then, if necessary, shaping the product. Compositions can be formulated into any of many possible dosage forms such as, but not limited to, tablets, capsules, liquid syrups, soft gels, suppositories, and enemas. A composition also can be formulated as a suspension in aqueous, non-aqueous or mixed media. Aqueous suspensions further can contain substances that increase the viscosity of the suspension including, for example, sodium carboxymethylcellulose, sorbitol, and/or dextran. Formulations can be sterilized if desired, provided that the method of sterilization does not interfere with the effectiveness of the inhibitor(s) contained in the formulation.

The invention will be further described in the following examples, which do not limit the scope of the invention described in the claims.

### EXAMPLES

### Example 1

### Ex Vivo Induction of Tolerance by Anti-CD40L

A bulk MLR was prepared by combining donor CD4+T cells obtained from C57BL/6 (H-2^{b}) mice (National Institutes of Health, Bethesda, Md.) in a 1:1 ratio with T cell-depleted, irradiated splenocytes obtained from bm12 (H-2^{bm12}), MHC class II disparate mice (The Jackson Laboratory, Bar Harbor, Me.). An anti-CD40L monoclonal antibody (hybridoma MR1, hamster IgG), obtained by culturing the hybridoma in 10% FBS/DMEM in an Accusyst Jr. hollow fiber bioreactor (Cellex Biosciences, Minneapolis, Minn.), was added to the bulk MLR culture and the cells were incubated for 7-10 days. Proliferative responses of the T cells to alloantigen were determined on days 2, 4, 5, 6, and 7 by measuring ³H-thymidine incorporation into newly synthesized DNA. The antibody removed from the cultures by washing three times with 2% FBS/PBS, and aliquots of the cells (0.3×10⁵, 10⁵, and 3×10⁵ cells) were administered in 0.5 mL DMEM by tail vein injection to bm12 mice subjected to a sublethal dose (6.0 Gy) of total body irradiation. Control irradiated mice received the same amounts of cells from MLR that had not been exposed to anti-CD40L. The mice were monitored for GVHD lethality, weight changes, and packed cell volume (PCV). Cells remaining from the primary MLR were mixed with fresh bm12 splenocytes for a secondary MLR, and proliferative responses to allogen were measured on days 1 through 5. Aliquots of the secondary MLR cultures were subsequently administered to irradiated bm12 mice.

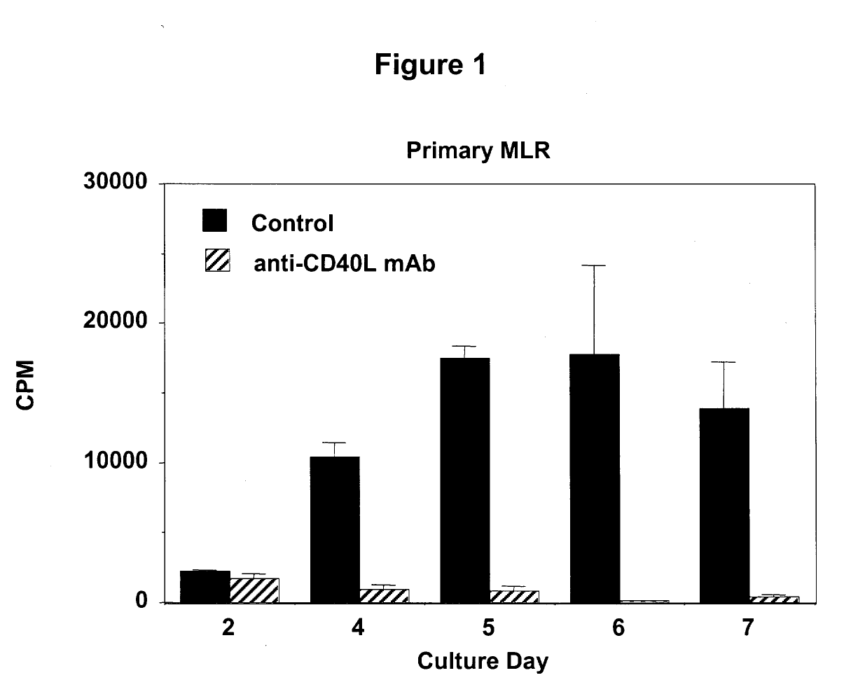

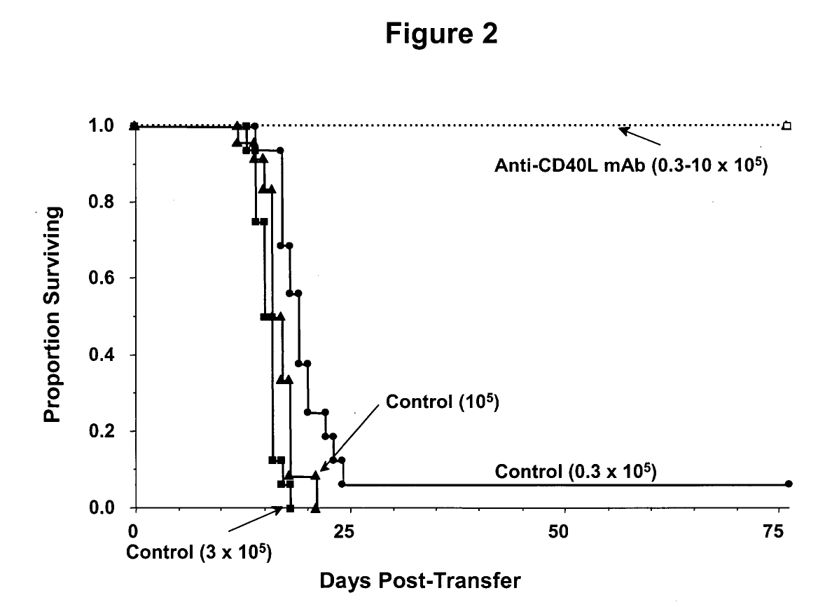

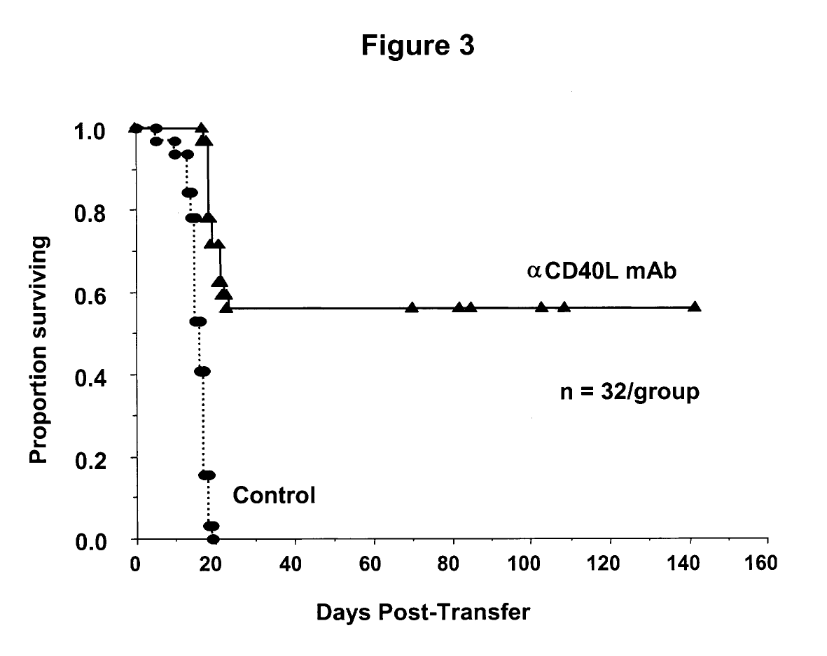

On all culture days examined, the proliferative response of cells in the primary MLR treated with anti-CD40L was greatly decreased as compared to the response of untreated cells (FIG. 1). Similar results were obtained with the secondary MLR cultures. Seventy-five days post-transfer, all mice survived in the groups that had been injected with primary MLR cells exposed to anti-CD40L (FIG. 2). In contrast, all mice in the groups injected with the two higher amounts of untreated MLR cells were dead by 25 days post-transfer, as were all but one of the mice injected with the lowest amount of untreated MLR cells. Similarly, nearly 60 percent of the mice injected with anti-CD40L treated cells from the secondary MLR survived at 160 days post-transfer, while all control mice were dead by 20 days post-transfer (FIG. 3). Thus, blocking the ability of CD40 ligand to interact with its APC-bound receptor can prevent GVHD in MHC class II disparate recipients.

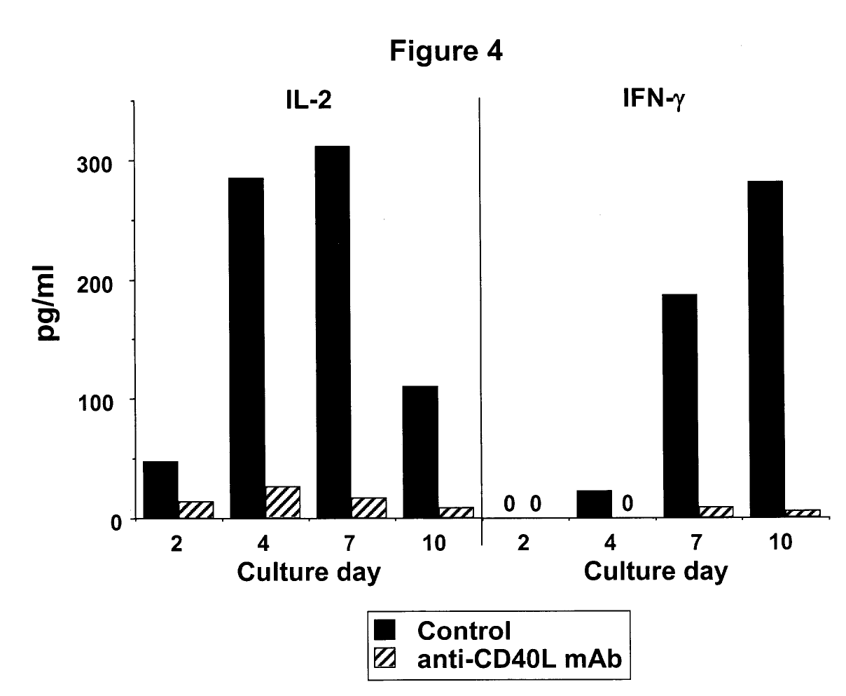

To determine whether tolerization affected cytokine production in the MLR cultures, levels of the Th1 cytokines IL-2 and interferon-gamma (IFN-γ) were measured on culture days 2, 4, 7, and 10 by enzyme-linked immunosorbant assay (ELISA; R&D Systems, Minneapolis, Minn.). Levels of both cytokines were significantly reduced on all days tested in the MLR cultures treated with anti-CD40L antibody as compared to untreated cultures (FIG. 4). The same effect was observed in secondary MLR cultures tested on days 1, 3, and 5. Thus, production of Th1 cytokines was reduced by inhibition of the CD40 ligand-receptor interaction.

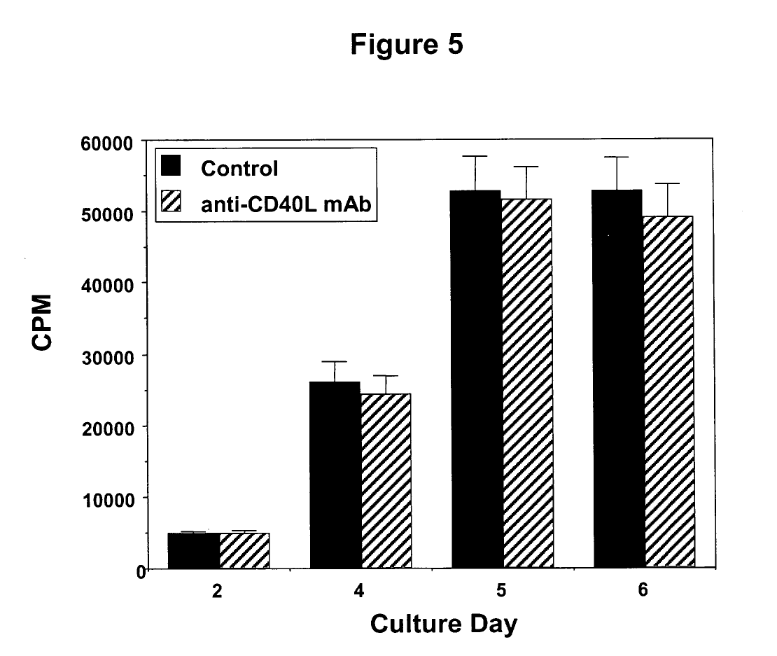

The decrease in IL-2 may be responsible for the loss of proliferative response in MLR cultures exposed to anti-CD40L antibody. To test this hypothesis, 50 IU/mL exogenous IL-2 was added to primary and secondary MLR, and the proliferative response to alloantigen was examined. Treatment with IL-2 restored the proliferative response to normal levels on all days tested in both primary MLR cultures (FIG. 5) and secondary MLR cultures.

### Example 2

### Ex Vivo Blockade of NF-κB Signaling in Alloreactive T Cells

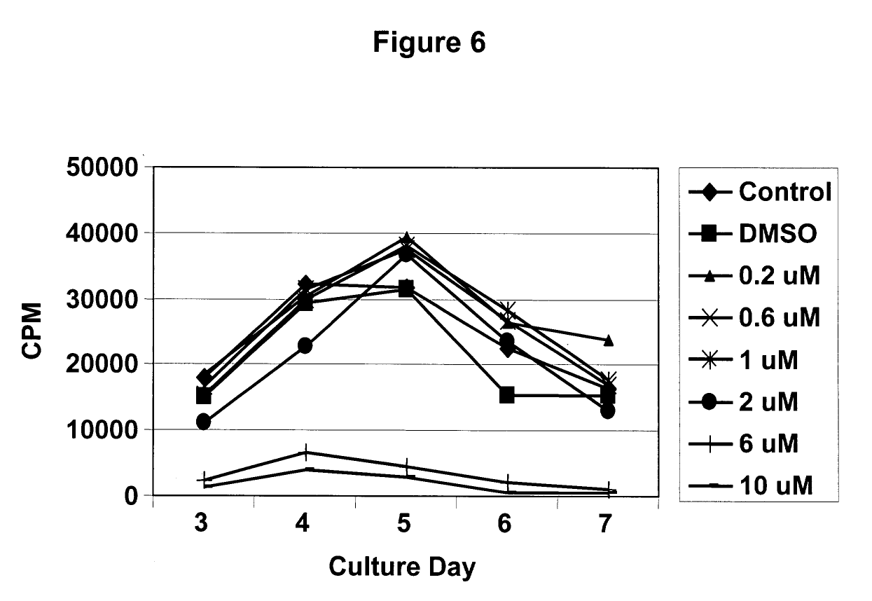

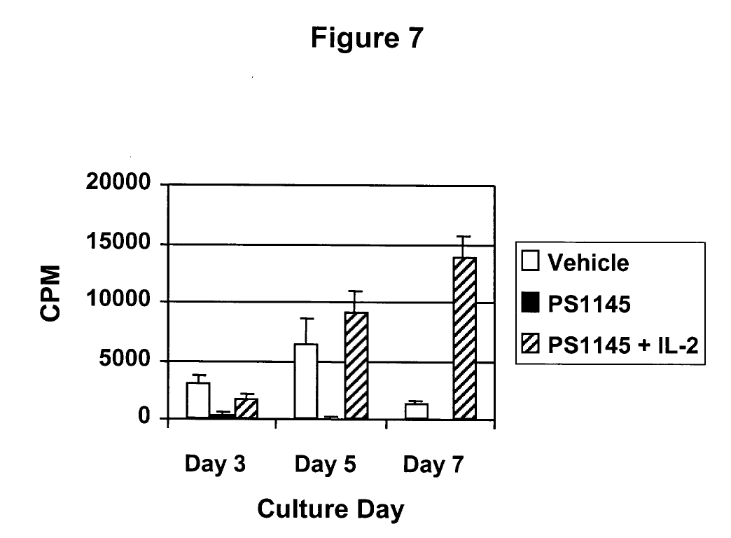

An MLR culture was prepared consisting of a 1:1 ratio of CD4+T cells obtained from C57BL/6 mice and T cell-depleted, irradiated MHC class II-disparate stimulators obtained from bm12 mice. PS1145, a potent inhibitor of IκB kinase and thus NF-κB activation, was added to the MLR culture at concentrations of 0.2 μM, 0.6 μM, 1 μM, 2 μM, 6 μM, and 10 μM. Control MLR cultures were left untreated or were treated with DMSO. T cell hyporesponsiveness to alloantigen was observed as a reduced level of proliferation in MLR treated with 6 μM and 10 μM PS1145 (FIG. 6). This hyporesponsiveness persisted after washing of the T cells and re-exposure to alloantigen. As with anti-CD40L treated MLR, alloantigen hyporesponsiveness was reversed by addition of 50 U/ml exogenous IL-2 (FIG. 7).

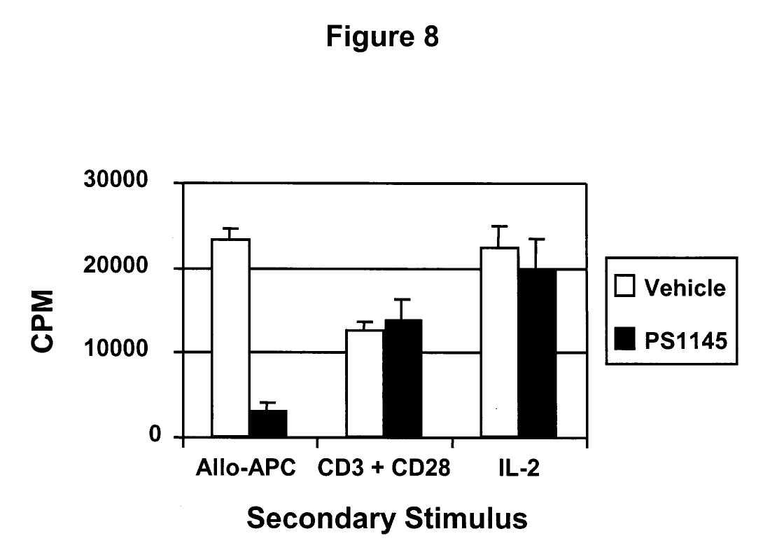

The tolerance induced by PS1145 was specific to alloantigen, as responses to non-specific mitogens remained largely intact. MLR cultures that were treated with 10 μM PS1145 and then re-exposed to allogeneic APC remained hyporesponsive (FIG. 8). In contrast, T cells in MLR cultures exposed to a secondary stimulus of CD3+ and CD28+cells exhibited proliferation at levels comparable to controls, as did PS1145-treated MLR exposed to exogenous IL-2. Other experiments using an MLR system containing a 1:1 ratio of alloreactive and non-alloreactive transgenic T cells indicated that treatment with 10 μM PS1145 increased the rate of T cell apoptosis selectively in alloreactive cells, as measured by Annexin V binding.

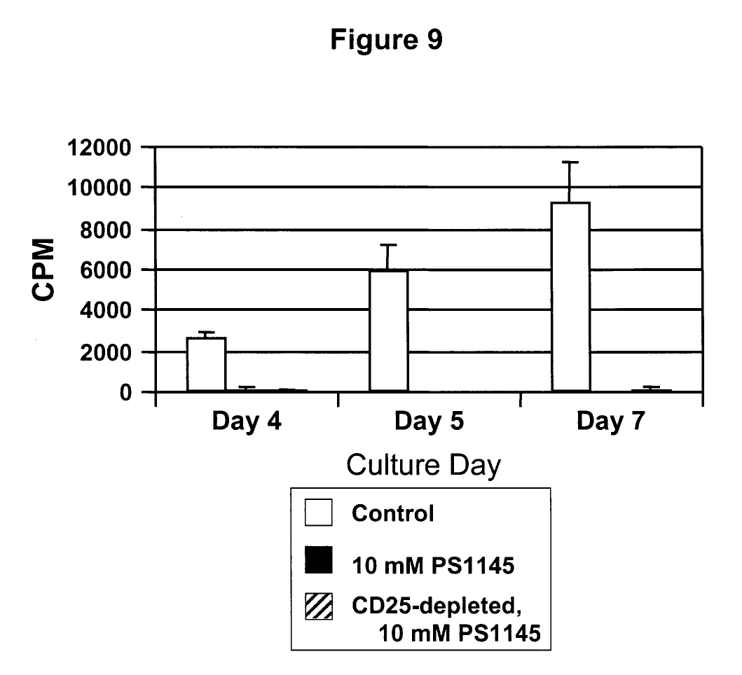

CD4⁺CD25⁺ cells were required in cultures tolerized by anti-CD40L. To determine whether CD25+T cells, which express the low-affinity IL-2 receptor α chain (IL-2Rα), were required for PS1145 induction of tolerance, CD25⁺-depleted T cells were used in primary and secondary MLR. In these experiments, hyporesponsiveness to alloantigen at days 4, 5, and 7 post-treatment with PS1145 was similar to that observed with non-depleted T cell populations (FIG. 9). Similarly, CD4⁺ CD25⁺ T cells were not required for tolerance induction by PS1145 in secondary cultures. Thus, PS1145 treatment directly induced tolerance in the absence of CD4⁺CD25⁺ cells, in contrast to their required presence in cultures tolerized by anti-CD40L.

CD4⁺ T cells that were allowed to recover from a vehicle-treated 7 day MLR were uniformly fatal upon adoptive transfer into sublethally irradiated MHC class-II disparate recipients, whereas T cells subjected to ex vivo PS1145 treatment were lethal to only 22% of recipients. Mice were intravenously injected with either 10⁵ naïve T cells, 10⁵ cells from 7 day control MLR or 10⁵ cells from MLR exposed to 6 μM or 10 μM PS1145 for 7 days. As shown in Table 1, no mice injected with naïve T cells or control MLR survived more than 90 days. In contrast, long-term survival was exhibited by five of eight mice injected with MLR cells exposed to 6 μM PS1145, and by nine of ten mice injected with MLR cells exposed to 10 μM PS1145. Thus, the NF-κB pathway is a critical regulator of T cell responses, and provides an ex vivo approach to inducing alloantigen-specific tolerance as a means of preventing GVHD.

TABLE 1
Long term survival (>90 days)
Donor CD4⁺ T Cell Treatment

| Naive | Control MLR | PS1145 MLR | Dose |
|---|---|---|---|
| 0/4 | 0/8 | 5/8 | 6 μM |
| 0/10 | 0/10 | 9/10 | 10 μM |
| 0/14 (0%) | 0/18 (0%) | 14/18 (78%) | |

### Example 3

### In Vivo Effects of PS1145

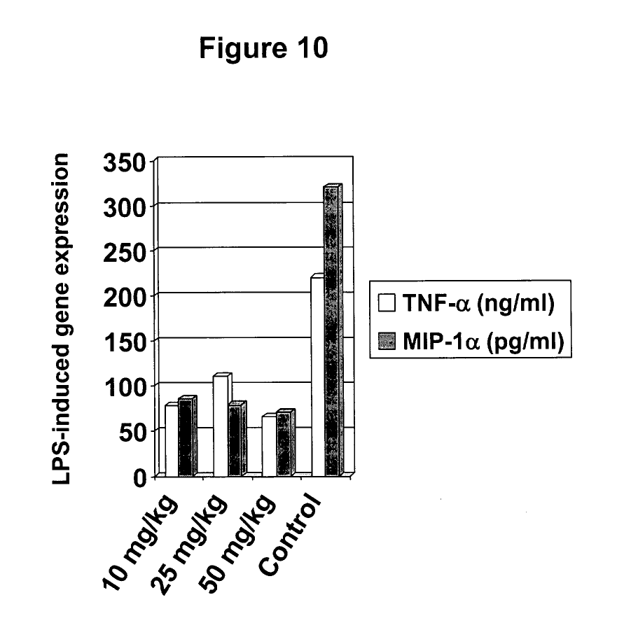

B6 and BALB/c mice were given 1 μg/kg of LPS, after which they orally received either control methylcellulose or PS1145 in methylcellulose. Animals were evaluated for expression of the cytokine TNF-α and the chemokine MIP-1α at 24-72 hours post-treatment, as assessed by ELISA. Expression of both genes was markedly reduced after treatment with 10 mg/kg, 25 mg/kg, or 50 mg/kg PS1145 as compared to expression in controls (FIG. 10).

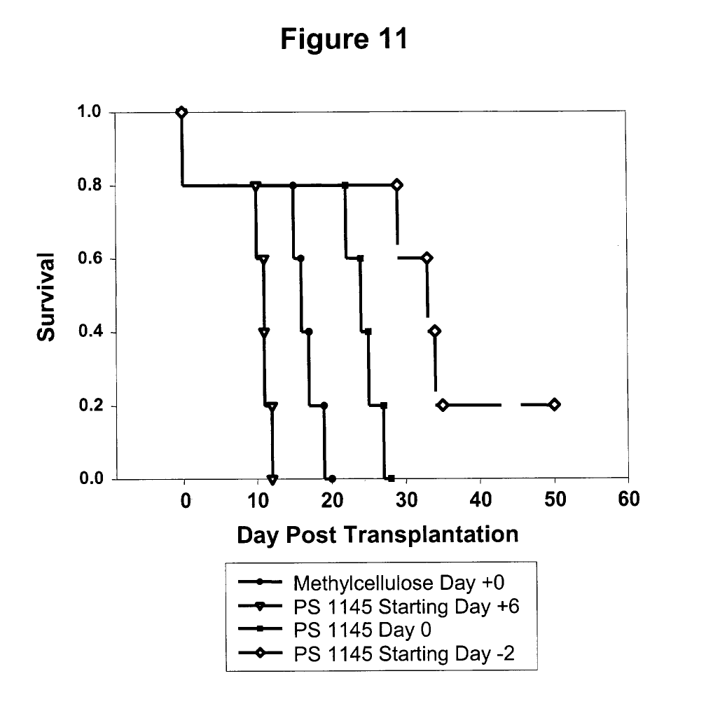

A murine stem cell transplant model was developed in which bone marrow was removed from B6 (H2^{b}) donors. The marrow was depleted of T cells by incubation with an anti-Thy 1.2 monoclonal antibody coupled to magnetic beads, followed by removal of T cells that bound the antibody using a magnetic column. Thy 1.2⁺ T cells were isolated from splenocytes using a similar approach and were added back to the bone marrow. Animals received between 1-5×10⁶ T cells and 3×10⁶ bone marrow cells after lethal irradiation. The T cell-depleted marrow was transplanted into B6D2H2^{bxd} recipient mice that had been subjected to a sublethal dose of irradiation (8.5 Gy). Recipient mice were divided into six groups, three of which received 1 mg PS1145 starting on day −2, day 0, or day 6 relative to transplant. The other three groups received methylcellulose as a control, starting on day −2, 0, or 6. PS1145 suspended in methylcellulose or methylcellulose alone was administered daily by gavage, through day 14. Mice were evaluated every three days for weight loss, GVHD, and outcome. As shown in FIG. 11, all animals treated with methylcellulose or with PS1145 starting on day 0 or day 6 were dead by post-transplant day 30, whereas about 20 percent of the mice that had received PS1145 starting on day −2 survived at least through day 50. Treatment with PS1145 that started on day 6 was correlated with an earlier death (all mice were dead by about day 12) as compared to treatment that started on day 0 (all mice were dead by about day 28). All mice in the methylcellulose control group were dead by day 20.

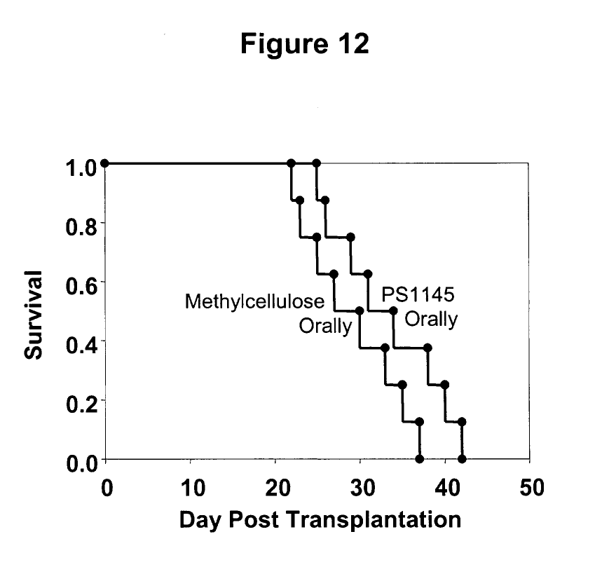

In a similar experiment, recipient mice were subjected to 1125 cGy of irradiation and then treated with oral doses of 5 mg/kg methylcellulose or PS1145. In this case, however, all animals in both groups were dead by about day 42 post-transplant (FIG. 12). There was little difference in survival time between the two groups. Thus, the use of PS1145 to ameliorate GVHD via an oral route was not effective using very heavy doses of irradiation.

Experiments also were performed in which 5 mg/kg PS1145 or methylcellulose was given intraperitoneally (i.p.) to mice starting on day −2 and ending on day 14 post-transplantation. B6D2 mice that had received 850 cGy of irradiation were injected with 3×10⁶ bone marrow cells from B6 donors and 5×10⁶ selected B6 T cells. As shown in Table 2 below, treatment with PS1145 resulted in the survival of 50% of B6D2 mice, compared to 0% of the control animals. Thus, i.p. treatment with PS1145 appeared to be more effective than oral treatment (compare Table 2 with FIGS. 11 and 12).

TABLE 2
Long-term survival following i.p. treatment

| Survival | PS1145 | Methylcellulose |
|---|---|---|
| Day 30 | 7/10 | 4/10 |
| Day 60 | 5/10 | 0/10 |

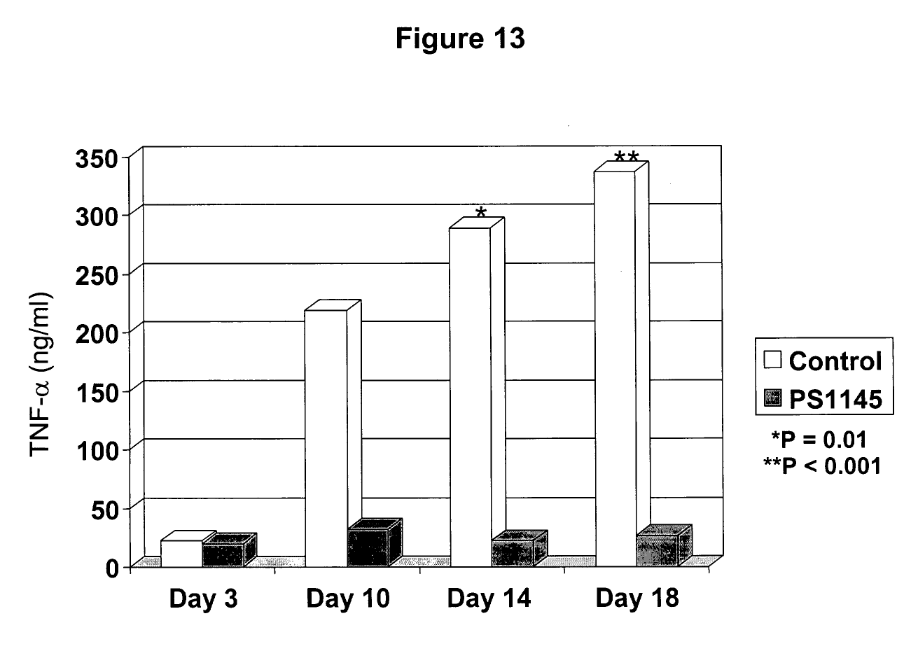

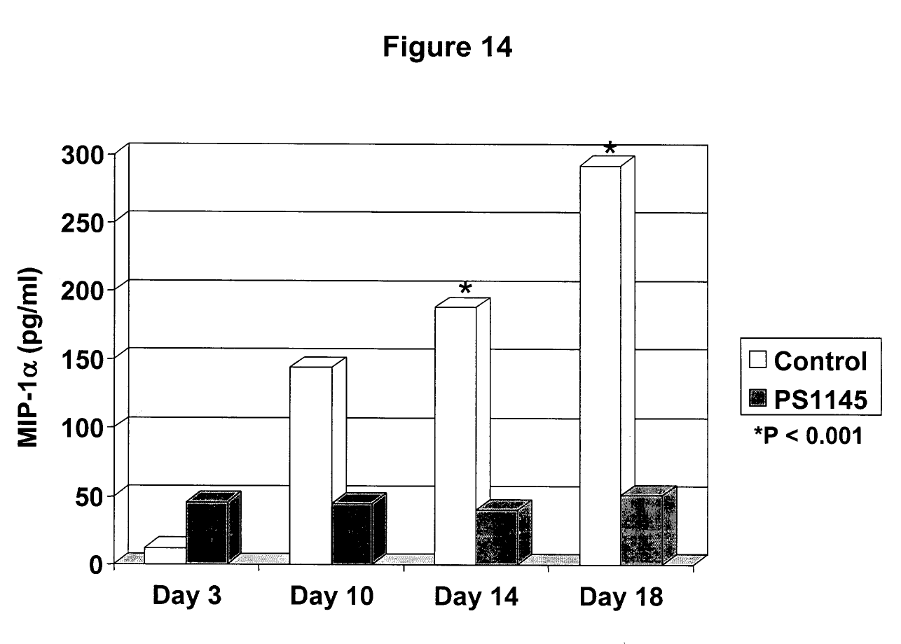

Expression of TNF-α and MIP-1α was evaluated in mice treated i.p. with PS1145 and subjected to transplantation of T cell-depleted bone marrow with Thy 1-selected splenocytes as a source of T cells. Mice in the test group received 5 mg/kg PS1145 on days −1 and −2 relative to transplant, while control animals were untreated. Animals were sacrificed from days 3-18 for assessment of TNF-α levels in the colon and small intestine and MIP-1α levels in the liver. A cytokine bead assay was used to measure levels of these factors. As shown in FIG. 13, TNF-α expression was significantly decreased following treatment with PS1145. While TNF-α levels in control animals increased from day 3 to day 18, the level was relatively constant in animals exposed to PS1145. Analogous results were obtained when MIP-1α levels in the liver were examined (FIG. 14). Thus, in vivo treatment with PS1145 resulted in decreased cytokine and chemokine expression.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for administering hematopoietic stem cells to a transplant recipient, said method comprising:
(a) providing a donor hematopoictic stem cell composition comprising non-autologous T cells;
(b) contacting said composition with an inhibitor of NF-κB activation to induce hyporesponsiveness of said T cells; and
(c) administering said contacted composition to said recipient, wherein said contacted composition has a lower GVHD potential than a corresponding non-contacted composition.

2. The method of claim 1, wherein said non-autologous T cells are alloreactive T cells.

3. The method of claim 1, wherein said inhibitor of NF-κB activation is PS1145.

4. A method for administering hematopoietic stem cells to a transplant recipient, said method comprising administering to said recipient (a) an inhibitor of NF-κB activation, and (b) a donor hematopoietic cell composition comprising alloreactive T cells.

5. The method of claim 4, wherein said inhibitor of NF-κB activation is PS1145.

6. The method of claim 4, wherein said inhibitor is administered prior to said transplant.

7. The method of claim 4, wherein said inhibitor is administered concurrent with said transplant.

8. The method of claim 4, wherein said inhibitor is administered after said transplant.

9. The method of claim 4, wherein said inhibitor is administered orally.

10. The method of claim 4, wherein said inhibitor is administered parenterally.

11. The method of claim 4, wherein said recipient has cancer.

12. The method of claim 11, wherein said cancer results in a solid tumor.

13. The method of claim 11, wherein said cancer is a hematopoietic cancer.

14. The method of claim 4, wherein said recipient has a bone marrow failure syndrome.

15. The method of claim 4, wherein said recipient has an inherited disorder.

16. A method of making a T cell tolerized to an alloantigen, said method comprising providing an isolated T cell, and contacting said isolated T cell with said alloantigen and an inhibitor of NF-κB activation.

17. A method for treating an autoimmune disorder in a subject, said method comprising identifying a subject having said autoimmune disorder, and administering to said subject a composition comprising a direct inhibitor of IKK.

18. The method of claim 17, wherein said inhibitor is PS1145.

19. A method for reducing transplant rejection, said method comprising administering to a transplant recipient a composition comprising a direct inhibitor of IKK, wherein said transplant is an organ transplant.

20. The method of claim 19, wherein said inhibitor is PS1145.
